(19) United States
(12) Patent Application Publication
(10) Pub. No.: US 2023/0218397 A1
(43) Pub. Date: Jul. 13, 2023

(54) DEVICES, SYSTEMS AND METHODS FOR PREVENTING PROLAPSE OF NATIVE CARDIAC VALVE LEAFLETS

(71) Applicant: 4C Medical Technologies, Inc., Maple Grove, MN (US)

(72) Inventors: Jeffrey W. Chambers, Maple Grove, MN (US); Jeffrey R. Stone, Minnetonka, MN (US)

(21) Appl. No.: 18/100,738

(22) Filed: Jan. 24, 2023

Related U.S. Application Data
(63) Continuation of application No. 16/429,874, filed on Jun. 3, 2019, now abandoned.
(60) Provisional application No. 62/680,073, filed on Jun. 4, 2018.

(51) Int. Cl. A61F 2/24

(52) U.S. Cl. CPC A61F 2/2454; A61F 2/2418; A61F 2230/0071; A61F 2250/0039; A61F 2250/001

(57) ABSTRACT

A collapsible and expandable prosthetic heart valve stent is provided and comprising an outer section, a valve support defining a flow channel therethrough, a transition section configured to smoothly transition the outer section to the valve support. The valve support is disposed within an interior defined by the outer section, with the inflow end of the valve support disposed inside the outer section's interior. In some cases, the outflow end of the valve support is at least partially defined by the transition section. The prosthetic leaflets are disposed on the inner surface of the valve support's flow channel and are located at or above the annulus of the heart chamber. A prolapse prevention system is attached to the stent to mitigate native valve leaflet prolapse.

### CROSS-REFERENCE TO RELATED APPLICATIONS

This application is a continuation of and claims the benefit of U.S. patent application Ser. No. 16/429,874, filed on Jun. 3, 2019 and entitled DEVICES, SYSTEMS AND METHODS FOR PREVENTING PROLAPSE OF NATIVE CARDIAC VALVE LEAFLETS, which claims the benefit of U.S. Provisional Application Ser. No. 62/680,073, filed Jun. 4, 2018 and entitled DEVICES, SYSTEMS AND METHODS FOR PREVENTING PROLAPSE OF NATIVE CARDIAC VALVE LEAFLETS, the contents of which are all hereby incorporated herein by reference in their entirety.

### STATEMENT REGARDING FEDERALLY SPONSORED RESEARCH OR DEVELOPMENT

Not Applicable

### BACKGROUND OF THE INVENTION

### Field of the Invention

The invention relates to devices, systems and features for mitigating paravalvular leak and optimizing functional efficiency of the prosthetic heart valve, including prosthetic mitral valve implant and prosthetic tricuspid valve implant. More specifically, mitigation of paravalvular leakage for a prosthetic mitral valve implant is provided.

### Description of the Related Art

The human heart comprises four chambers and four heart valves that assist in the forward (antegrade) flow of blood through the heart. The chambers include the left atrium, left ventricle, right atrium and right ventricle. The four heart valves include the mitral valve, the tricuspid valve, the aortic valve and the pulmonary valve. See generally FIG. 1.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood into the chamber it just flowed out of For example, the mitral valve when working properly provides a one-way valving between the left atrium and the left ventricle, opening to allow antegrade flow from the left atrium to the left ventricle and closing to prevent retrograde flow from the left ventricle into the left atrium. This retrograde flow, when present, is known as mitral regurgitation or mitral valve regurgitation.

FIG. 2 illustrates the relationship between the left atrium, annulus, chordae tendineae and the left ventricle relative to the mitral valve leaflets. As is shown, the upper surface of the annulus forms at least a portion of the floor or lower surface of the left atrial chamber, so that for purposes of description herein, the upper surface of the annulus is defined as marking the lower boundary of the left atrial chamber.

Native heart valves may be, or become, dysfunctional for a variety of reasons and/or conditions including but not limited to disease, trauma, congenital malformations, and aging. These types of conditions may cause the valve structure to fail to close properly resulting in regurgitant retrograde flow of blood from the left ventricle to the left atrium in the case of a mitral valve failure. FIG. 3 illustrates regurgitant blood flow with an exemplary dysfunctional mitral valve.

Mitral valve regurgitation is a specific problem resulting from a dysfunctional mitral valve that allows at least some retrograde blood flow back into the left atrium from the right atrium. In some cases, the dysfunction results from mitral valve leaflet(s) that prolapse up into the left atrial chamber, i.e., above the upper surface of the annulus instead of connecting or coapting to block retrograde flow. This backflow of blood places a burden on the left ventricle with a volume load that may lead to a series of left ventricular compensatory adaptations and adjustments, including remodeling of the ventricular chamber size and shape, that vary considerably during the prolonged clinical course of mitral regurgitation.

Regurgitation can be a problem with native heart valves generally, including tricuspid, aortic and pulmonary valves as well as mitral valves.

Native heart valves generally, e.g., mitral valves, therefore, may require functional repair and/or assistance, including a partial or complete replacement. Such intervention may take several forms including open heart surgery and open heart implantation of a replacement heart valve. See e.g., U.S. Pat. No. 4,106,129 (Carpentier), for a procedure that is highly invasive, fraught with patient risks, and requiring not only an extended hospitalization but also a highly painful recovery period.

Less invasive methods and devices for replacing a dysfunctional heart valve are also known and involve percutaneous access and catheter-facilitated delivery of the replacement valve. Most of these solutions involve a replacement heart valve attached to a structural support such as a stent, commonly known in the art, or other form of wire network designed to expand upon release from a delivery catheter. See, e.g., U.S. Pat. Nos. 3,657,744 (Ersek); 5,411,552 (Andersen). The self-expansion variants of the supporting stent assist in positioning the valve, and holding the expanded device in position, within the subject heart chamber or vessel. This self-expanded form also presents problems when, as is often the case, the device is not properly positioned in the first positioning attempt and, therefore, must be recaptured and positionally adjusted. This recapturing process in the case of a fully, or even partially, expanded device requires re-collapsing the device to a point that allows the operator to retract the collapsed device back into a delivery sheath or catheter, adjust the inbound position for the device and then re-expand to the proper position by redeploying the positionally-adjusted device distally out of the delivery sheath or catheter. Collapsing the already expanded device is difficult because the expanded stent or wire network is generally designed to achieve the expanded state which also resists contractive or collapsing forces.

Besides the open heart surgical approach discussed above, gaining access to the valve of interest is achieved percutaneously via one of at least the following known access routes: transapical; transfemoral; transatrial; and transseptal delivery techniques.

Generally, the art is focused on systems and methods that, using one of the above-described known access routes, allow a partial delivery of the collapsed valve device, wherein one end of the device is released from a delivery sheath or catheter and expanded for an initial positioning followed by full release and expansion when proper positioning is achieved. See, e.g., U.S. Pat. Nos. 8,852,271 (Murray, III); 8,747,459 (Nguyen); 8,814,931 (Wang); 9,402,720 (Richter); 8,986,372 (Murray, III); and 9,277,991 (Salahieh); and U.S. Pat. Pub. Nos. 2015/0272731 (Racchini); and 2016/0235531 (Ciobanu).

In addition, all known prosthetic heart valves are intended for full replacement of the native heart valve. Therefore, these replacement heart valves, and/or anchoring or tethering structures, physically extend out of the left atrial chamber, in the case of mitral valves, and engage the inner annulus and/or valve leaflets, in many cases pinning the native leaflets against the walls of the inner annulus, thereby permanently eliminating all remaining functionality of the native valve and making the patient completely reliant on the replacement valve. In other cases, the anchoring structures extend into the left ventricle and may anchor into the left ventricle wall tissue and/or the sub-annular surface at the top of the left ventricle. Others may comprise a presence in, or engagement with, a pulmonary artery.

Obviously, there will be cases when native valve has lost virtually complete functionality before the interventional implantation procedure. In this case the preferred solution will comprise an implant that does not extent outside of, e.g., the left atrium, and that functions to completely replace the native valve function. However, in many other cases, the native valve remains functional to an extent and may, or may not, continue to lose functionality after the implantation procedure. A preferred solution in this case comprises delivery and implantation of a valve device that will function both as a supplemental or augmentation valve without damaging the native leaflets in order to retain native valve leaflet functionality as long as present, while also being fully capable of replacing the native function of a valve that slowly loses most or all of its functionality post-implantation of the prosthetic valve.

Further, as seen in FIG. 2, the annular surface comprises an irregular landscape with commissures and other elevation changes and/or shaping that differ from person to person. Accommodation of these anatomical features would be advantageous.

Finally, known prosthetic cardiac valves consist of two or three leaflets that are arranged to act as a one-way valve, permitting fluid flow therethrough in the antegrade direction while preventing retrograde flow. The native mitral valve is located retrosternally at the fourth costal cartilage, consisting of an anterior and posterior leaflet, chordae tendinae, papillary muscles, ventricular wall and annulus connected to the atria. Each native leaflet is supported by chordae tendinae that are attached to papillary muscles which become taut with each ventricular contraction preserving valvular competence. Both the anterior and posterior leaflets of the native valve are attached via primary, secondary and tertiary chordae to both the antero-lateral and posterio-medial papillary muscles. A disruption in either papillary muscle in the setting of myocardial injury, can result in dysfunction of either the anterior or posterior leaflet of the mitral valve. Other mechanisms may result in failure of one, or both of the native mitral leaflets. In the case of a single mitral valve leaflet failure, the regurgitation may take the form of a non-central, eccentric jet of blood back into the left atrium. Other leaflet failures may comprise a more centralized regurgitation jet. Known prosthetic valve replacements generally comprise leaflets which are arranged to mimic the native valve structure, which may over time become susceptible to similar regurgitation outcomes.

Known implantable prosthetic valves may be improved upon by employing structures that may extend the functionality of native valve leaflets in a supplement first, replace when required structure as described herein. It would be highly advantageous in this regard to provide a structure that only engages prolapsing native leaflets at a point of coaptation, thus preventing prolapse.

Certain inventive embodiments described herein are readily applicable to single or two chamber solutions, unless otherwise indicated. Moreover, certain embodiments discussed herein may be applied to preservation and/or replacement of native valve functionality generally, with improved native leaflet coaptation and/or prolapsing, and are not, therefore, limited to the mitral valve and may be extended to include devices and methods for treating the tricuspid valve, the aortic valve and/or pulmonary valves.

Various embodiments of the several inventions disclosed herein address these, inter alia, issues.

### BRIEF DESCRIPTION OF THE SEVERAL VIEWS OF THE DRAWINGS

FIG. 1 illustrates certain features of the heart in cross-section.

FIG. 2 illustrates a cross-sectional perspective view of the left side of the heart.

FIG. 3 illustrates a cross-sectional view of the heart showing retrograde blood flow resulting from mitral valve regurgitation compared with normal blood flow.

FIG. 4 illustrates and end view of one embodiment of a valve support.

FIG. 5 illustrates a perspective view of one embodiment of a prosthetic heart valve stent device.

FIG. 6 illustrates a partial cross-sectional view of one embodiment of a prolapse prevention system of the present invention.

FIG. 7 illustrates a partial cross-sectional view of one embodiment of a prolapse prevention system of the present invention.

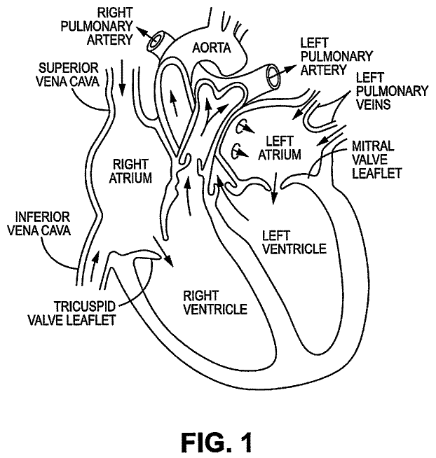

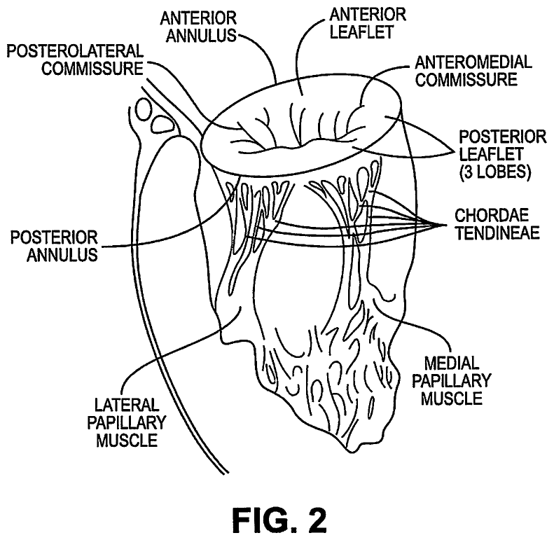

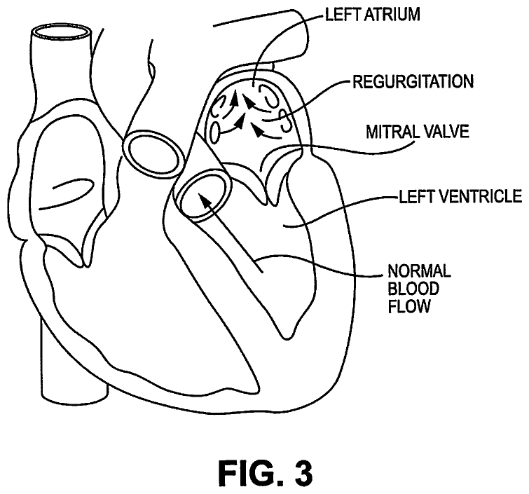

### DETAILED DESCRIPTION OF THE INVENTION

Generally, various embodiments of the present invention are directed to devices and methods for creating optimal apposition of a support structure or stent of a prosthetic heart valve to treat cardiac mitral or tricuspid valve regurgitation, mitigating paravalvular leak and optimizing functional efficiency of the prosthetic heart valve.

The support structure (i.e. stent) has multiple features that function to aid with the treatment of cardiac valve regurgitation (mitral or tricuspid). These functions include its function as a scaffold for the functioning prosthetic valve leaflets of the current invention, apposition to the atrial anatomy, optimized radial force for compliance with atrial distension, ability to load and deploy from a minimally invasive delivery system, and geometry to support with mitigating against paravalvular leak (PVL). The design features of the stent are adapted to meet one or more of the functions identified above. Specific design features and attributes for the stents are discussed in detail below.

The stent design concepts are intended to support minimally invasive procedures for the treatment of valvular regurgitation — mitral, tricuspid and/or otherwise. The stents may be self-expandable (e.g. nitinol or similar materials) or balloon expandable (e.g. cobalt chromium or similar materials). The stents are made of cells that may be open celled diamond like structures or continuous structures that have a working cell element. The stents may also be constructed using tubing, wires, braids or similar structures. Specific design features that aid with the functioning of the stent are described in detail below.

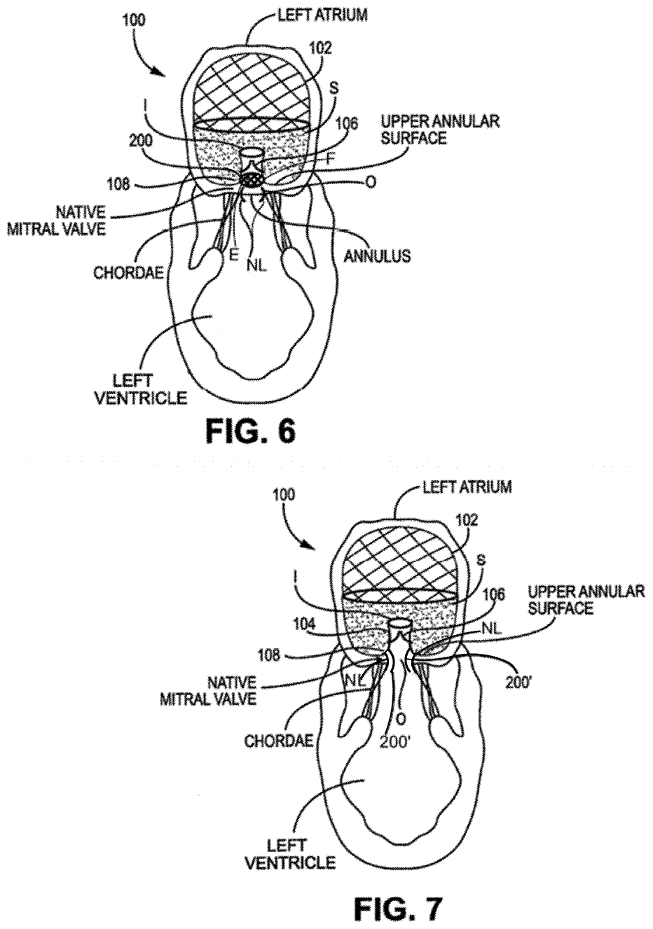

Referring to FIG. 5, an expandable and collapsible prosthetic heart valve stent device 100 may comprise an outer section 102—that may generally look like a ball when undeformed and fully expanded and an inner valve support section 104, adapted to support and retain prosthetic valve leaflets 106, shown in FIGS. 6 and 7, within the inner valve support section 104, most preferably at a point that is located above the native annulus, and spaced away or above the native leaflets, as shown in FIGS. 6 and 7, though other attachment points for the prosthetic leaflets 106 are within the scope of the present invention. Inner valve support 104 may be cylindrical, but in a preferred embodiment may also be at least partially conical, with a larger diameter at an outflow end 0 than the diameter across at least portions of an inflow end I, wherein the inflow end I is disposed radially inside the outer frame section and wherein the outflow end 0 may define a lower end or edge of the valve support 104. Thus, in a purely conical arrangement, the valve support section 104 may comprise a smoothly decreasing diameter thereacross and this smooth diameter decrease may extend from the outflow end O to the inflow end I. In other embodiments, the inflow end I may comprise one or more lobes extending radially outwardly and that interrupt the smooth conical profile. A preferred embodiment in this regard provides one lobe for each prosthetic leaflet 106 attached within the inner valve support 104 to allow for fuller freedom of movement and improved coaptation.

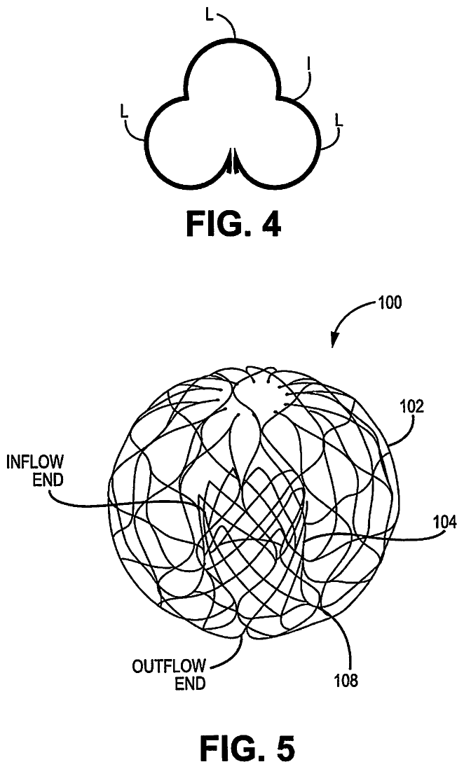

FIG. 4 illustrates an end view of one embodiment of an inflow end I of the inner valve support 104 comprising three lobes L. See also commonly assigned US patent application 62/612,836, filed Jan. 2, 2018, the disclosure of which is hereby incorporated in its entirety.

A preferred construction comprises the prosthetic leaflets 106 disposed or spaced above the native leaflets when the prosthetic heart valve stent device 100 is implanted, wherein the prosthetic leaflets 106 are attached and spaced sufficiently away from (above) the native leaflets so as to not physically interfere or interact with the native leaflets. However, certain embodiments contemplate some interaction with the native leaflets.

The layer of stent cells that transition from the outer section to the inner section of the stent are termed as transition cells forming a transition section 108 generally as illustrated in FIG. 5.

The outer and inner sections of the stent may be constructed from one continuous structure or may combine two or more structures to achieve intended design goals. As known in the art, stent structures may be formed using complementary shaped mandrels, including the outer section 102 of the stent, the transition section 108, and the inner valve support 104—including lobes L discussed above in certain embodiments- as a single unitary structure.

Referring now to FIG. 6, an exemplary prosthetic heart valve stent device 100 is shown implanted within the left atrium. The prosthetic valve leaflets 106 are shown as elevated above the upper annular surface of the left atrium and generally located or centered above the annulus between the left atrium and left ventricle and comprises a prolapse prevention structure 200. An exemplary screen or mesh prolapse prevention structure 200 is shown that extends across the interior flow channel defined by the inner valve support 102 from inflow end Ito outflow end O and covers at least partially the native annulus. The screen or mesh prolapse prevention structure 200 may be connected to, or integrated with, the outflow end of the inner valve support 104 and/or may be connected generally at an inner edge of the transition section 108. The exemplary prolapse prevention structure 200 of FIG. 6 is positioned at a point axially relative to the native leaflets NL and their normal healthy functional coaptation point so as to engage the native leaflets NL at that coaptation point to prevent as much regurgitation as possible using the native leaflets NL and without unnecessarily engaging the supra-annularly positioned prosthetic leaflets 106 for that task, while preventing prolapse of the native leaflets NL.

In certain embodiments the prolapse prevention structure 200 may comprise a coating comprising anti-thrombus formation compound(s) and/or anti-endothelization compound(s).

The exemplary prolapse prevention structure 200 of FIG. 6 may comprise a plurality of round or flat spanning elements or sections E that extend across and engage an outer support structure F that is engaged with the implanted prosthetic valve structure as described above. Outer support structure F may be shaped and sized to fit the shape and size of the portion of the inner valve support 102 to which it is connected or integrated with.

In certain embodiments, the outer support structure F may be positioned generally so that it engages with tissue and works to prevent paravalvular leakage (PVL). For example, the outer support structure of the prolapse prevention structure may engage, or be integrated with, the transition section described above to provide a barrier against PVL.

Further in this regard, a preferred embodiment of the device shown in the Figures comprises a skirt S, comprising fabric or tissue, disposed along a portion of the outer surface of the outer frame element 102 and that extends along the outer surface of the transition section 108 and along the inner surface, or inwardly facing surface, of the inner valve support 102 so that the skirt S is facing the flow channel defined therein from the inflow end Ito the outflow end 0. In certain embodiments, the outer support structure F of the prolapse prevention structure 200 may also be covered with a fabric or tissue that, in combination with the tissue engagement of the outer frame and transition elements, may assist in preventing PVL.

Alternatively, the spanning elements or sections E may be integrated with the prosthetic heart valve stent device 100 as described above without aid or requirement of an outer support structure.

Generally the spanning elements or sections E may be disposed transverse to the blood flow through the inner valve support 104. In the case of an outer support structure F, the spanning elements or sections E may be substantially coplanar with the outer support structure F or, alternatively may extend either upwardly or downwardly from the outer support structure F. All such exemplary structures are acceptable so long as the native leaflets NL are engaged by at least one spanning element or section E at a point of normal healthy functional coaptation. Thus, the outer support structure F may be located at a point above the normal coaptation point, wherein at least one spanning element or section extends therebelow to the normal coaptation point.

Turning now to FIG. 7, another exemplary embodiment of a prolapse prevention structure 200′ is illustrated. Here, instead of a circular support structure, two leaf guards 200′ are positioned as aligned with the two native leaflets of the mitral valve and having a distal end that is positioned to engage the native leaflets NL. The leaf guards 200′ may be connected to, or integrated with the inner valve support 104 or may be connected to, or integrated with, the transition section 108. In this embodiment, the leaf guards 200′ are disposed at or below the upper annular surface and reach a distance into the annulus. In some embodiments the leaf guards 200′ are arranged so as to not engage the native leaflets until they reach the point of normal healthy coaptation.

Alternatively, the leaf guards 200′ may effectively pin the native leaflets as shown in FIG. 7, so that the implanted prosthetic valve device 100 immediately becomes a full replacement device. The leaf guards 200′ of FIG. 7 are shown as comprising a semi-circle, wherein an upstream end of each of the leaf guards 200′ is connected within the inner surface of the inner valve support 102 at the outflow end 0 of the inner valve support 102. The leaf guards 200′ are radially spaced from each other around the inner valve support 102 to enable the leaf guards 200′ to align with, and engage, the two native leaflets when the device is implanted into the patient's right atrium. Leaf guards 200′ in FIG. 7 each extend from the inner valve support 102, in the downstream direction along the annulus and comprise an inner portion that effectively curves or wraps around the native mitral valve annulus to engage the native leaflets. The inner portion of the leaf guards 200′ of FIG. 7 comprise a slightly smaller radius than the outer portion of the leaf guards 200′. The outer, larger radius, portion of the leaf guards 200′ is exposed to blood flow within the mitral valve annulus.

As shown in FIG. 7, and in equivalent embodiments, the leaf guards 200′ may be configured to provide a positioning and locating function that allows alignment of the inner valve support 104 and prosthetic leaflets 106 held therein over the native annulus and native leaflets during implantation and subsequent operation. In this regard, the leaf guards 200′ may function in two key ways: prolapse prevention or pinning of the leaflets, and alignment, locating and positioning of the prosthetic heart valve stent device 100 when implanted.

An alternative embodiment to individual leaf guards 200′ may comprise one or more semi-circular leaf guard extensions, of a number and at a position that comports with the number of native leaflets and their general position. These semi-circular leaf guards 200′ may be positioned to (1) only engage native leaflets at the normal healthy coaptation point; or (2) effectively pin the native leaflets. In either case, the semi-circular leaf guards 200′ may be configured to, as described above, assist with positioning, locating and aligning the prosthetic valve device relative to the annulus. Further, as discussed above, the semi-circular leaf guards 200′ may be at least partially covered with a tissue or fabric that may be coextensive or connected with the skirt material of the outer frame, transition section and/or inner valve to assist in preventing PVL.

A still more alternative embodiment may comprise a leaf guard extension comprising an unbroken structure extending from the inner valve support 104 and/or transition section 108, the unbroken structure taking an expanded shape that may be substantially circular and/or may substantially match the shape of the annulus. Again, the unbroken leaf guard extension may be configured to, as described above, assist with positioning, locating and aligning the prosthetic heart valve stent device 100 relative to the annulus and may either engage the native leaflets NL at a normal healthy coaptation point or may work to pin the native leaflets NL. Further, as discussed above, the unbroken leaf guard extension may be at least partially covered with a tissue or fabric that may be coextensive or connected with the skirt material of the outer frame 102, transition section 108 and/or inner valve support 104 to assist in preventing PVL.

In certain embodiments, at least a lower portion of the leaf guards 200′ may be relatively flexible and responsive to the pressure and fluid flow changes, while prevented from flexing upwardly past a coaptation point. Thus, during systole, the lower portion of the leaf guards 200′ may be urged to extend radially inwardly to engage the rising native leaflets at a normal healthy coaptation point. During diastole, the lower portion of the leaf guards 200′ may be urged to substantially straighten or be otherwise positioned to allow blood flow therealong such as may be seen with reference to FIG. 7.

In some cases, the prolapse prevention structure 200 of FIG. 6, and the prolapse prevention structure comprising the leaf guards 200′ of FIG. 7 may be constructed to provide a second prosthetic valve, wherein they may, in response to the pressure and fluid flow changes discussed above, effectively open and close at least partially to further assist in preventing regurgitation. In this way, the prolapse prevention structure 200 of FIG. 6, and the prolapse prevention structure comprising the leaf guards 200′ of FIG. 7 may be viewed as the initial regurgitation barrier, in combination with the native leaflets, with the prosthetic leaflets functioning to stop any additional regurgitant. If, or when, the native leaflet function deteriorates nearly completely, the combination of the prolapse preventing structure 200 of FIG. 6, or the prolapse prevention structure comprising the leaf guards 200′ of FIG. 7, and the prosthetic leaflets 106 may work together to form a two-stage prosthetic staged set. This staging of regurgitant flow stoppage may work to extend the life of the native leaflets.

It is noteworthy that the various embodiments of the presently described prosthetic valve stent device 100 may be delivered percutaneously via one of at least the following known access and delivery routes: femoral access, venous access, trans-apical, trans-aortic, trans-septal, and trans-atrial, retrograde from the aorta delivery techniques. Alternatively, the prosthetic valve stent device 100 may be delivered and implanted using surgical and/or open heart techniques.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A collapsible and expandable stent for implanting into a right atrium of a patient's heart comprising:
an expanded ball-shaped outer section comprising an outer surface, an inner surface, and defining an interior;
an inner valve support extending upward into the interior of the outer section and comprising an inflow end and an outflow end, wherein the inflow end is superior to the outflow end when the stent is implanted, the inner valve support comprising an inner surface defining a flow channel between the inflow and outflow ends, the inner valve support positioned entirely within the interior of the outer section;
a plurality of prosthetic valve leaflets disposed within the flow channel defined by the inner valve support section, wherein the prosthetic valve leaflets are configured to allow flow from the inflow end to the outflow end of the flow channel and prevent flow from the outflow end of the flow channel to the inflow end of the flow channel;
a collapsible and expandable transition section comprising a plurality of cells extending between the expanded ball-shaped outer section and the inner valve support, wherein the inner valve support extends radially upward into the interior of the outer section, the transition section comprising an outer surface and an inner edge that faces the interior defined by the expanded ball-shaped outer section, wherein the expanded ball-shaped outer section, the transition section and the inner valve support are a single unitary stent structure formed of a continuous series of stent cells; and
two semi-circular leaf guards comprising an upstream and a downstream end, wherein each semi-circular leaf guard is connected at the upstream end with the inner surface of the valve support at an outflow end of the valve support, wherein the semi-circular leaf guards are radially spaced away from each other, wherein each leaf guard extends away from the valve support in a downstream direction,
wherein the plurality of prosthetic valve leaflets are disposed and spaced away in an upstream direction from the two semi-circular leaf guards.

2-6. (canceled)

7. A collapsible and expandable stent for implanting into at least one chamber of a patient's heart comprising:
an outer section comprising an outer surface, an inner surface, and defining an interior;
a valve support extending radially upward into the interior of the outer section and comprising an inflow end and an outflow end, the inflow end extending radially upward into the outer section, the valve support comprising an inner surface defining a flow channel between the inflow and outflow ends, the valve support positioned entirely within the interior of the outer section;
a plurality of prosthetic valve leaflets disposed within the flow channel defined by the valve support section, wherein prosthetic valve leaflets are configured to allow flow from the inflow end to the outflow end of the flow channel and prevent flow from the outflow end of the flow channel to the inflow end of the flow channel;
a collapsible and expandable transition section comprising a plurality of cells extending between the outer section to the valve support, wherein the valve support extends radially upward into the interior of the outer section, the transition section comprising an outer surface and an inner edge that faces the interior defined by the outer section; and
a prolapse prevention structure comprising a plurality of round or flat spanning elements connected to, or integrated with, with the stent and configured to extend across the flow channel defined by the inner valve support.

8. The stent of claim 7, wherein the plurality of round or flat spanning elements are connected to, or integrated with, the outflow end of the inner valve support.

9. The stent of claim 7, wherein the plurality of round or flat spanning elements are connected to, or integrated with, the inner edge of the transition section.

10. The stent of claim 7, wherein the plurality of round or flat spanning elements are disposed transverse to a flow of blood through the flow channel defined by the inner valve support.

11. The stent of claim 7, wherein at least one of the round or flat spanning elements are adapted to engage native leaflets of a native valve within the at least one chamber of the patient's heart only at point where normal healthy coaptation would normally occur.

12. The stent of claim 7, wherein the plurality of round or flat spanning elements comprises a screen or mesh.

13. (canceled)

14. The stent of claim 1, wherein the outflow end of the inner valve support does not extend outwardly past the transition section.

15-20. (canceled)
